(19) United States
(12) Patent Application Publication
(10) Pub. No.: US 2016/0317580 A9
(43) Pub. Date: Nov. 3, 2016

(54) COMPOSITION AND METHOD FOR DENTAL REMINERALIZATION

(71) Applicant: Robert L. Karlinsey, Indianapolis, IN (US)

(72) Inventor: Robert L. Karlinsey, Indianapolis, IN (US)

(21) Appl. No.: 13/711,001

(22) Filed: Dec. 11, 2012

Related U.S. Application Data
(63) Continuation-in-part of application No. 12/018,627, filed on Jan. 23, 2008, now Pat. No. 8,556,553.

(51) Int. Cl. A61K 33/42

(52) U.S. Cl. CPC A61K 33/42

(57) ABSTRACT

A dental remineralization composition, including a calcium phosphate portion and an organic portion. The calcium phosphate portion is selected from the group including β-TCP and combinations thereof and the β-TCP portion is present in concentrations between about 0.5 ppm to about 10,000 ppm. The organic portion is selected from the group including aspartic acid, glutamic acid, polypeptides, polymers containing functional groups, and combinations thereof and the organic portion is percent in concentrations between about 0.5 ppm to about 10,000 ppm.

### CROSS REFERENCE TO RELATED APPLICATIONS

This application is a continuation-in-part of and claims priority to co-pending utility patent application Ser. No. 11/701,210 filed Jan. 31, 2007 and published as U.S. Patent Pub. No. 2007/0178220

### TECHNICAL FIELD

The novel technology relates generally to the field of chemistry, and, more particularly, to a method and composition for remineralizing teeth and dentition.

### BACKGROUND

Even through fluoride applications and other preventive measures, dental decay still affects the majority of the world's population. For instance, the United States National Health and Nutrition Examination Survey and the World Health Organization reports that dental decay is on the rise, especially in children. Fluoride confers clinically-proven and significant dental benefits, yet additional benefits are clearly in demand. Complimentary attempts in improving dental health include combining fluoride with calcium phosphate, the latter of which have varying degrees of solubility and remineralization characteristics when applied to weakened enamel.

Thus, one non-invasive and potentially high patient-compliant and acceptable approach in combating dental decay may be the use of calcium phosphate salts or minerals. Compared to calcium, saliva manifests relatively high phosphate content, and phosphate can be commonly found in acidic beverages and foodstuffs (e.g. such as dairy and vegetable products). Additionally, public perception of calcium is very positive. Skeletal health, for instance, is maintained with sufficient calcium intake through bone modeling processes. For women in particular, calcium is vital to maintaining proper bone strength, especially during pregnancy or after menopause. In terms of dental health, calcium is critically important to the preservation of the tooth structure but can easily be compromised. Therefore, it is not unusual that food and nutrition companies incorporate calcium into beverages, foodstuffs, vitamins, etc.

Typically, conventional calcium phosphates can compromise bioavailable fluoride, and therefore, therapeutic efficacy. This is largely due to the reactivity of bioavailable fluoride and calcium, which rapidly form calcium fluoride when introduced together in an aqueous environment prior to or during an oral hygienic event. Therefore, if one improves fluoride efficacy through the addition of a calcium phosphate agent without compromising bioavailable calcium or fluoride, enhanced dental health benefits may be realized.

### SUMMARY

A novel approach is needed to cheaply and easily extend the benefits of fluoride without compromising fluoride bioavailability. The following novel technology constitutes a simple combination of low levels of unmodified beta-tricalcium phosphate (β-TCP) with or without organic molecules comprising, for example carbonyl, carboxyl, carboxylate, sulfonyl, phosphoric, hydroxyl, or amino functional groups. The organic molecules include vitamins, proteins, peptides, surfactants and especially amino acids. The organic molecules also include polymers of the small molecule. Of particular interest are aspartic acid and glutamic acid, which are water-soluble and negatively charged at neutral pH conditions. Additionally, amino acids can be assembled into polypeptides and are involved in cellular behavior and tissue formation. β-TCP is chosen because it is a precursor in the formation of apatite, the principle mineral phase in teeth and bone. Additionally, it is biocompatible, bioactive, and it is sparingly soluble. Thus, low levels of β-TCP can be added to dental formulations containing fluoride and applied to the teeth via rinsing, toothbrushing, etc to provide anti-erosive, anti-caries, anti-hypersensitivity, and other dental health benefits.

### DETAILED DESCRIPTION

For the purposes of promoting an understanding of the principles of the novel technology and presenting its currently understood best mode of operation, reference will now be made to the embodiments illustrated in the drawings and specific language will be used to describe the same. It will nevertheless be understood that no limitation of the scope of the novel technology is thereby intended, with such alterations and further modifications in the illustrated device and such further applications of the principles of the novel technology as illustrated therein being contemplated as would normally occur to one skilled in the art to which the novel technology relates.

Calcium phosphate minerals, such as alpha tricalcium phosphate (α-TCP), beta tricalcium phosphate (β-TCP), hydroxyapatite (HAP), dicalcium phosphate, calcium sulfate, octacalcium phosphate, amorphous calcium phosphate, calcium chloride, and the like play an important role the in the constitution of teeth and bone. However, they are not typically coupled with fluoride in a single compartment aqueous format due to the undesirable formation of insoluble calcium fluoride that directly compromises fluoride-bioavailability and therapeutic efficacy.

Amino acids comprise proteins, which are intimately involved in tissue construction and repair. Previously, arginine, an amino acid, has been incorporated into toothpaste for improved relief of dental hypersensitivity. However, amino acids have not been previously coupled with a calcium phosphate mineral in a fluoride-based dental vehicle to provide anti-erosion, anti-caries, anti-hypersensitivity, and other dental health benefits. Therefore a significant opportunity exists in coupling the two components to create a fluoride compatible system that imparts favorable dental health benefits.

Our approach is the simple combination of unmodified ‘as is’ amino acid and β-TCP ingredients into an aqueous solution or water-based mixture typically having pH greater than 3.5. This combination encourages the interfacing between the sparingly soluble β-TCP and the negatively charged COO⁻ functional group of the amino acid. Fluoride can then be added to the system if desired. The resultant combination offers an especially economical approach in achieving superior dental health benefits, since no synthesis, extraction, separation, or similar chemical or physical process is required to generate the calcium phosphate-amino acid complex. This may be especially useful in over-the-counter oral pharmaceutical formulations, where raw material costs are particularly important.

Typically the weight fraction of β-TCP can range between 1% and 100% and especially between 40% and 90%. The weight fraction of the amino acid can range between 5% to 99% and especially between 10% and 60%. The family of amino acids includes ariginine (C₆H₁₄N₄O₂), histidine (C₆H₉N₃O₂), lysine (C₆H₁₄N₂O₂), serine (C₃H₇NO₃), theronine (C₄H₉NO₃), asparagine (C₄H₈N₂O₃), glutmine (C₅H₁₀N₂O₃), cysteine (C₃H₇NO₂S), selenocysteine (C₃H₇NO₂Se), glycine (C₂H₅NO₂), proline (C₅H₉NO₂), alanine (C₃H₇NO₂), isoleucine (C₆H₁₃NO₂), leucine (C₆H₁₃NO₂), methionine (C₅H₁₁NO₂S), phenylalanine (C₉H₁₁NO₂), tryptophan (C₁₁H₁₂N₂O₂), tyrosine (C₉H₁₁NO₃), valine (C₅H₁₁NO₂), aspartic acid (C₄H₇NO₄), and glutamic acid (C₅H₉NO₄). Although amino acids are discussed explicitly in this application, polypeptides and other organic molecules manifesting active functional groups can also be used in the same weight fraction. These include but are not limited to vitamin molecules such as, for example, ascorbic acid, tocopherol, calciferol, and pyridoxal phosphate. Additionally, negatively charged surfactants such as sodium lauryl sulfate (chemical formula: C₁₂H₂₅SO₄Na) and neutral polymers such as polyethylene glycol (chemical formula: HOCH₂—(CH₂OCH₂)ₙ—CH₂OH, where n typically ranges between 9 and 30) may also be used. Additionally, organic molecules manifesting at least one of the following groups including an amide (CON), amine (NH₂), nitrate (ONO₂), phosphate (OPO₃), sulfonyl (SO₂), sulfo (SO₃), sulfate (SO₄), carbonyl (CO), carboxylate (COO⁻), carboxyl (COOH), or peroxy (OO) group.

The technology is firstly created by mixing β-TCP and the amino acid in a single compartment with or without a solvent, preferably water. In the absence of water, the agents can be shaken with as little force as shaking by hand for several minutes. If desired, the agents can also be mixed together with low-speed tumblers or paddle mixers to improve homogeneity. If combined with water to the appropriate volume desired, calcium phosphate and amino acid can be added separately and then shaken to improve homogeneity of the resultant suspension. The presence of water, or another polar solvent that is generally recognized as safe for human use, is useful for interfacing between the calcium phosphate and amino acid. At a pH above 3.7, for instance, aspartic acid, having chemical formula HOOCCH(NH₂)CH₂COOH, deprotonates to create an anionic functional group. If fluoride is to be added to the formulation for dental benefits, preferably it is added after introduction of β-TCP and amino acid.

The above technology is to be implemented into dental formulations, such as toothpastes, mouth rinses, gels, varnishes, mints, gums, pastes, foam, etc. Therefore, above technology can be combined with surfactants, humectants, preservatives, fluoride, flavoring systems, pigment and color systems, binders, emulsifiers, resins, water, non-water solvents, abrasives, etc. Since teeth are naturally porous, there are opportunities for promising mineralizing agents to repair weakened enamel and prevent mineral loss.

The examples below demonstrate the compatibility of the β-TCP plus amino acid system with fluoride, as well as the remineralization efficacy in the presence and absence of fluoride.

### EXAMPLES

### Example 1

### Fluoride Compatibility

This first example summarized in Tables 1 and 2 demonstrates the compatibility between fluoride and calcium phosphate-amino acid systems in a single aqueous compartment under accelerated aging conditions. Fluoride compatibility was evaluated based on the ratio between β-TCP and the amino acid, the type of amino acid, and the fluoride level. Reductions less than 10% of the Control (i.e. Theoretical) fluoride level are consistent with the Food and Drug Administration's monograph requirements for fluoride-containing oral pharmaceuticals.

TABLE 1
Fluoride compatibility after accelerated aging conditions for 91 days at
40° C. Measurements made in triplicate with fluoride-sensitive
electrode. ASP refers to aspartic acid.

| | | | | % Reduction vs |
|---|---|---|---|---|
| System | % NaF | Theoretical [F⁻] | Measured [F⁻] | Control |
| 225 ppm F + 36 ppm β- | 0.05% | 225 ppm | 211.6 ± 2.0 ppm | −6.0% |
| TCP + 4 ppm ASP | | | | |
| 225 ppm F + 72 ppm β- | 0.05% | 225 ppm | 200.9 ± 3.0 ppm | −10.7% |
| TCP + 8 ppm ASP | | | | |
| 500 ppm F + 36 ppm β- | 0.11% | 500 ppm | 486.7 ± 5.0 ppm | −2.7% |
| TCP + 4 ppm ASP | | | | |
| 1100 ppm F + 90 ppm β- | 0.24% | 1100 ppm | 1115.0 ± 16.5 ppm | +1.4% |
| TCP + 10 ppm ASP | | | | |

TABLE 2
Fluoride compatibility after accelerated aging conditions for 97 days at 40° C.
Measurements made in triplicate with fluoride-sensitive electrode. ASP refers to
aspartic acid. GLU refers to glutamic acid.

| | | | | % Reduction vs |
|---|---|---|---|---|
| System | % NaF | Theoretical [F⁻] | Measured [F⁻] | Control |
| 225 ppm F + 36 ppm β-TCP + 36 ppm | 0.05% | 225 ppm | 218.1 ± 2.2 ppm | −3.1% |
| ASP | | | | |
| 225 ppm F + 72 ppm β-TCP + 72 ppm | 0.05% | 225 ppm | 207.7 ± 2.6 ppm | −7.7% |
| ASP | | | | |
| 225 ppm F + 108 ppm β-TCP + | 0.05% | 225 ppm | 198.3 ± 1.4 ppm | −11.9% |
| 108 ppm ASP | | | | |
| 1100 ppm F + 90 ppm β-TCP + 90 ppm | 0.24% | 1100 ppm | 1110.6 ± 11.7 ppm | +1.0% |
| ASP | | | | |
| 1100 ppm F + 180 ppm β-TCP + | 0.24% | 1100 ppm | 1057.4 ± 13.0 ppm | −3.9% |
| 180 ppm ASP | | | | |
| 1450 ppm F + 90 ppm β-TCP + 90 ppm | 0.32% | 1450 ppm | 1422.3 ± 16.8 ppm | −1.9% |
| ASP | | | | |
| 225 ppm F + 36 ppm β-TCP + 36 ppm | 0.05% | 225 ppm | 228.9 ± 18.2 ppm | +1.7% |
| GLU | | | | |
| 225 ppm F + 72 ppm β-TCP + 72 ppm | 0.05% | 225 ppm | 204.9 ± 6.8 ppm | −8.9% |
| GLU | | | | |
| 1100 ppm F + 90 ppm β-TCP + 90 ppm | 0.24% | 1100 ppm | 1101.4 ± 9.4 ppm | +0.1% |
| GLU | | | | |
| 1100 ppm F + 180 ppm β-TCP + | 0.24% | 1100 ppm | 1080.6 ± 7.7 ppm | −1.8% |
| 180 ppm GLU | | | | |
| 1450 ppm F + 90 ppm β-TCP + 90 ppm | 0.32% | 1450 ppm | 1459.5 ± 15.0 ppm | +0.7% |
| GLU | | | | |

The data above demonstrate low levels of β-TCP and amino acids can be added to fluoride without significantly compromising fluoride bioavailability. The data above are provided as a guide to the formulation of oral pharmaceuticals containing water and various fluoride levels and should not be restrictive to that shown in the Tables.

### Example 2

### Remineralization of Weakened Enamel with Fluoride

This second example details the laboratory evaluation when β-TCP and amino acids are combined with fluoride to treat eroded enamel. It is customary to use laboratory models, such as pH cycling studies that alternate between the strengthening and weakening of teeth using solutions, suspensions, slurries, etc. that emulate the clinical setting. The protocol is briefly discussed below. Three groups of enamel specimens were subjected to a remineralization/demineralization pH cycling model that included three 2-minute treatment periods and five 2-minute acid challenge (1% citric acid, pH=3.8) periods. In between these events, the specimens were immersed in a 50:50 mixture of pooled human saliva and artificial saliva. The pooled human saliva was collected from healthy volunteers. The cycling regimen was repeated for a total of 20 days, with interim microhardness measurements made after 10 days of cycling. The treatment and saliva systems were magnetically agitated at 300 rpm, while the acid challenge was static. After each treatment and acid challenge, the specimens were rinsed with DI water prior to placement into the saliva mixture, which was changed once daily after the third acid challenge. Examination of enamel strength was then performed using Vickers surface microhardness. The results after 10 and 20 days of pH cycling is shown in Table 3.

TABLE 3
Summary of surface microhardness results. ASP refers to aspartic acid.

| Groups | [Ca²⁺] | VHN⁰ | VHN¹⁰ | ΔVHN¹⁰ | VHN²⁰ | ΔVHN²⁰ |
|---|---|---|---|---|---|---|
| Distilled Water (0 ppm F) | 0 ppm | 214.5 ± 2.8 | 258.3 ± 3.1 | 42.3 ± 3.3 | 216.9 ± 3.8 | 0.8 ± 4.6 |
| 225 ppm F (NaF) | 0 ppm | 215.1 ± 3.2 | 283.0 ± 6.0 | 67.8 ± 7.1 | 240.1 ± 6.0 | 25.0 ± 8.1 |
| 225 ppm F (NaF) + 36 ppm | 14.0 ppm | 214.1 ± 3.0 | 290.1 ± 4.2 | 76.0 ± 5.2 | 254.4 ± 5.6 | 40.4 ± 6.4 |
| β-TCP + 36 ppm | | | | | | |
| ASP | | | | | | |

VHN⁰ = mean baseline Vickers Hardness Number (VHN) ± SEM (N = 10);
VHN¹⁰ = mean VHN ± SEM (N = 10) after 10 days of cycling;
ΔVHN¹⁰ = difference between mean VHN¹⁰ ± SEM (N = 10) and VHN⁰ after 10 days of cycling;
VHN²⁰ = mean VHN ± SEM (N = 10) after 20 days of cycling;
ΔVHN²⁰ = difference between mean VHN²⁰ ± SEM (N = 10) and VHN⁰ after 20 days of cycling;

The data above are provided as an example of the dental benefits that can be achieved in a format containing water, fluoride, and the novel calcium phosphate-amino acid system. The remineralization of eroded enamel is clear with marked improvements over fluoride alone after 10 and 20 days of cycling. Additionally, the example above demonstrates the boost that the calcium phosphate-amino acid system provides to fluoride in the presence of saliva. The data and example applies to other dental health benefits, such as the repair of caries-like lesions and relief of dental hypersensitivity.

### Example 3

### Remineralization of Weakened Enamel without Fluoride

This third example details the pre-clinical evaluation when β-TCP and amino acids are combined with a sugar-free format and applied to weakened teeth having early caries-like lesions. Three groups of human enamel specimens were subjected to a remineralization/demineralization pH cycling model that included three 5-minute treatment periods and three 30-minute acid challenge (polyacrylic acid-lactic acid, pH=5.0) periods. In between these events, the specimens were immersed in artificial saliva. The treatments were comprised of either unflavored paraffin-stimulated saliva or expectorate from volunteers chewing on two sugarfree gummy bears (mass per gummy was 3.1 grams) immediately before the treatment was to be administered. Once expectorate was collected in a cup for each treatment for Group 3, 15.5 mg of β-TCP and 15.5 mg of aspartic acid were then added. After treatment events, the specimens were lightly brushed with a soft toothbrush under running distilled water to prevent the formation of deposits on the specimen surfaces. After each acid challenge the specimens were rinsed with distilled water prior to placement into the artificial saliva, which was changed once daily after the second treatment period. The cycling regimen was repeated for a total of 20 days. The treatment and saliva systems were magnetically agitated at 300 rpm, while the acid challenge was static.

After the cycling procedure, the specimens were sectioned in order to measure the longitudinal microhardness and determine the approximate size/density of the lesion (ΔZ). The data collected from this study are presented in Table 4 and reveal a significant subsurface benefit can be achieved by adding β-TCP and aspartic acid to the gummy expectorate. The subsurface strengthening is especially apparent at depths near 37.5 μm, and extends to depths at least to 150 μm. Thus, the addition of β-TCP facilitates repair of the weakened enamel. The size of the lesion, ΔZ, after 20 days of cycling is especially small for the specimens treated with of β-TCP relative to the other two control groups. This novel remineralization technology then appears especially promising and robust.

TABLE
Summary of cross-sectional microhardness results.

| Enamel | Knoops Hardness Number (KHN) | | |
|---|---|---|---|
| Depth | | Group 2: Saliva + | Group 3: Saliva + Two |
| (μm) | Group 1: Saliva | Two Gummis | Gummis + β-TCP + ASP |
| 12.5 | 40.6 ± 2.3 | 57.3 ± 5.4 | 38.9 ± 3.8 |
| 25 | 94.0 ± 7.3 | 101.7 ± 10.1 | 92.5 ± 11.8 |
| 37.5 | 195.0 ± 13.8 | 224.8 ± 21.6 | 245.5 ± 27.2 |
| 50 | 276.1 ± 12.8 | 317.7 ± 10.7 | 353.3 ± 16.4 |
| 75 | 312.9 ± 6.0 | 328.3 ± 9.6 | 334.1 ± 9.1 |
| 100 | 314.4 ± 5.3 | 312.2 ± 13.1 | 338.3 ± 9.7 |
| 125 | 316.1 ± 6.1 | 314.2 ± 10.1 | 351.5 ± 10.7 |
| 150 | 300.7 ± 7.7 | 316.4 ± 8.1 | 351.7 ± 6.2 |
| ΔZ | 747.3 ± 85.6 | 402.6 ± 117.2 | 270.1 ± 83.0 |

KHN = mean Knoops Hardness Number ± SEM (N = 10);
ΔZ = mean density of lesion ± SEM (N = 10).

Thus, low levels of calcium phosphates may improve dental benefits when combined with fluoride. Here, the combination unmodified β-TCP with amino acids and fluoride yields tooth benefits, such as remineralization, inhibition of demineralization, arrestment of caries/erosive progression, and the like. In particular, β-TCP levels in the range between 0.5 ppm (0.0005 wt. %) thru 2000 ppm (i.e. 0.2 wt. %) may work as well or even more effectively for fluoride concentrations between 0 and 5,000 ppm. For higher levels of fluoride, such as ranging between 5,000 through 25,000 ppm fluoride, β-TCP levels may increase up to 20,000 ppm (2 wt. %).

While the novel technology has been illustrated and described in detail in the drawings and foregoing description, the same is to be considered as illustrative and not restrictive in character. It is understood that the embodiments have been shown and described in the foregoing specification in satisfaction of the best mode and enablement requirements. It is understood that one of ordinary skill in the art could readily make a nigh-infinite number of insubstantial changes and modifications to the above-described embodiments and that it would be impractical to attempt to describe all such embodiment variations in the present specification. Accordingly, it is understood that all changes and modifications that come within the spirit of the novel technology are desired to be protected.

1. A dental remineralization composition, comprising:
a beta tricalcium phosphate portion; and
an organic portion;
wherein the beta tricalcium phosphate portion is present in concentrations between about 0.5 ppm to about 2,000 ppm; and
wherein the organic portion is selected from the group including aspartic acid, glutamic acid, polypeptides, polymers containing functional groups, and combinations thereof;
wherein the organic portion is percent in concentrations between about 0.5 ppm to about 10,000 ppm.

2. The composition of claim 1 wherein the organic portion is present in amounts ranging from about 5 ppm to 200 ppm.

3. The composition of claim 1 wherein the beta tricalcium phosphate portion is present in amounts ranging from about 5 ppm to 200 ppm.

4. The composition of claim 1 wherein the functional groups are selected from the including group including amides (CON), amines (NH₂), nitrates (ONO₂), phosphates (OPO₃), sulfonyls (SO₂), sulfos (SO₃), sulfates (SO₄), carbonyls (CO), carboxylates (COO⁻), carboxyls (COOH), peroxys (OO), and combinations thereof.

5-15. (canceled)

16. A dental remineralization composition, comprising:
a beta-tricalcium phosphate portion; and
a fluoride portion connected to the beta-tricalcium phosphate portion;
wherein the beta-tricalcium phosphate portion is present in concentrations between about 0.5 ppm to about 20,000 ppm; and
wherein the fluoride portion present in concentrations ranging up to about 25,000 ppm.

17. The dental remineralization composition of claim 16 wherein the fluoride portion is connected as a functional group to the beta-tricalcium phosphate portion.

18. The dental remineralization composition of claim 16 wherein the beta-tricalcium portion is particulate and wherein the beta-tricalcium phosphate has a particle size in the range from about 1 nanometer to about 5 microns.

19. A dental remineralization composition, comprising:
a dental delivery vehicle portion;
an amino acid functionalized beta-tricalcium phosphate portion dispersed in the dental delivery vehicle portion; and
a fluoride portion dispersed in the dental delivery vehicle portion;
wherein the amino acid functionalized beta-tricalcium phosphate portion is present in concentrations between about 0.5 ppm to about 20,000 ppm; and
wherein the fluoride portion present in concentrations ranging from about 1 and about 25,000 ppm.

20. The dental remineralization composition of claim 16 wherein the amino-acid functionalized beta-tricalcium portion is particulate and wherein the amino acid functionalized beta-tricalcium phosphate has a particle size in the range from about 1 nanometer to about 5 microns.

21. A dental remineralization composition, comprising:
a dental delivery portion;
an admixture dispersed in the dental delivery portion; and
a fluoride portion dispersed in the dental delivery portion;
wherein the admixture includes beta-tricalcium phosphate powder and amino acids;
wherein beta-tricalcium phosphate is present in the dental delivery portion in concentrations between about 0.5 ppm to about 20,000 ppm; and
wherein fluoride is present in the dental delivery portion in concentrations ranging from about 1 and about 25,000 ppm.

22. The dental remineralization composition of claim 16 wherein the beta-tricalcium portion is particulate and wherein the beta-tricalcium phosphate has a particle size in the range from about 1 nanometer to about 5 microns.
